(19) United States
(12) Patent Application Publication
(10) Pub. No.: US 2008/0260708 A1
(43) Pub. Date: Oct. 23, 2008

(54) METHODS AND RELATED SYSTEMS AND FORMULATIONS TO NORMALIZE AND IMPROVE HUMAN BODY CHEMISTRY AND HEALING ABILITY

(76) Inventor: Mickey A. Hall, Costa Mesa, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 11/737,740

(22) Filed: Apr. 19, 2007

(51) Int. Cl. A61K 38/48; A61K 33/44; A61K 36/53; A61K 36/906; A61K 36/81; A61K 36/28; A61K 31/375; A61K 31/198; A61K 31/455

(52) U.S. Cl. 424/93.45; 424/94.64; 424/760; 424/756; 424/125; 514/574; 424/745; 514/474; 424/722; 424/764; 514/561; 514/564; 514/54; 514/356

(57) ABSTRACT

Methods, systems and formulations for normalizing and improving human body chemistry and the body's natural ability to heal itself. In one embodiment a system including effective amounts of a digestive enzyme, soluble and insoluble fiber, laxative, probiotics, vitamin C, potassium, protease enzymes, lipase, lysine, taurine, proline, choline, inositol, inositol hexaphosphate, policosanol, charcoal, bentonite clay, thyme, ascorbic acid, magnesium citrate, calcium citrate, methylsulfonyl methane, cayenne pepper, magnesium, potassium, ester-c, ginger and niacin, lysine calcium, stevia leaf, citric acid, a tincture of bayberry bark, juniper berries, yam root, cramp bark, golden seal root, fennel seed, uva ursi leaves, ginger root, lobelia herb, catnip herb, and peppermint leaf, golden seal root, Echinacea angustifolia root, ginger root, and licorice root, a tincture of black walnut hulls, venus fly trap, chaparral, wormwood, licorice root, slippery elm, cloves and comfrey root, burdock root, sheep sorrel, rhubarb root, slippery elm, olive leaf and yarrow flower is provided.

### BACKGROUND

1. Field

The present invention generally relates to methods and related systems and formulations for improving health and wellbeing in a human. More specifically, the present invention is generally directed to methods and related systems and formulations that address specific parts of the human body to normalize human body chemistry and improve the body's own innate ability to heal itself.

2. General Background

Throughout human history, mankind has continually tried to develop better methods for providing relief from physical pain and illness. From the thousands of botanical extracts to the present day pharmaceutical preparations, it has always been a high priority for mankind to find the best treatments available for relief of the myriad of illnesses and their symptoms.

Unfortunately, mankind's attempts at providing relief from the myriad illnesses that plague mankind have focused primarily on treatment of the symptoms of the particular disease instead of its cause. Although the treatment of symptoms often provides a welcome relief from the pain and discomforts that are associated with the particular illness, it does not address the illness itself. Additionally, and more importantly, the focus of most present-day treatments that primarily attempt only to reduce or eliminate the symptoms associated with the illness, in most if not all instances, suppress the human body's natural self-cleansing efforts and ability to heal itself. Although rapid relief of the symptoms associated with a condition or illness is desirable for the individual being treated, its desirability is greatly reduced or illuminated when one appreciates the negative impact the treatment often has on complicated yet vital body chemistry and resolving the condition or illness itself through the body's innate self-healing capabilities. Therefore, the long-term side effect of a focus on treating symptoms and pain results in a permanent interference with the body's sophisticated chemistry and self-healing capabilities, which greatly increases the development of various forms of known degenerative diseases and conditions. Accordingly, there exists a need for systems and/or formulations that normalize and improve human body chemistry functions and the body's ability to heal itself. The present invention provides these and other related advantages.

### SUMMARY

In brief and general terms, the embodiments of the present invention include, but are not limited to methods, systems and formulations which act to normalize and/or improve various human body chemistry functions thereby supporting and enhancing the body's innate ability to self-heal and self-regulate to resolve or improve the condition of unwanted diseases and illnesses.

More specifically, in one embodiment of the present invention, a system for normalizing and improving human body chemistry and the body's natural ability to heal itself is provided. The system is comprised of effective amounts of a digestive enzyme, soluble and insoluble fiber, a laxative, one or more probiotics, vitamin C, potassium, one or more protease enzymes, lipase, lysine, taurine, proline, choline, inositol, inositol hexaphosphate, policosanol, charcoal, bentonite clay, thyme, ascorbic acid, calcium citrate, magnesium citrate, methylsulfonyl methane, magnesium, potassium, ester-c, ginger and niacin, lysine calcium, stevia leaf, citric acid, a tincture of bayberry bark, juniper berries, yam root, cramp bark, golden seal root, fennel seed, uva ursi leaves, ginger root, lobelia herb, catnip herb, and peppermint leaf, golden seal root, Echinacea angustifolia root, ginger root, and licorice root, a tincture of black walnut hulls, venus fly trap, chaparral, wormwood, licorice root, slippery elm, cloves and comfrey root, burdock root, sheep sorrel, rhubarb root, slippery elm, olive leaf and yarrow flower, which are to be consumed by a human to normalize and improve human body chemistry and the natural ability to heal itself.

In another embodiment of the present invention, the digestive enzyme is obtained from a plant and comprised of any combination of one or more of the following: amylase, protease, lipase, cellulase, maltase, lactase and sucrase enzymes, the soluble and insoluble fiber is comprised of organic flax seed, psyillium seed and husks and oat fibers, the mild herbal laxative is any combination of two or more of the following: senna leaf, cascara sagrada, aloe vera, buckhorn bark, rhubarb root, hyssop leaf and fenugreek seed, the probiotic consists of any combination of five of more of following bacteria or yeast: lactobacillus acidophilus, bifidobacteria bifidum, lactobacillus sporogenes, lactococcus lactus, lactococcus cremoris, lactobacillus kefir, leuconostoc cremoris, lactobacillus caucasicus, lactobacillus plantarum, lactobacillus salivarius, lactobacillus bulgaricus, lactobacillus helveticus, bifidobacterium infantis, bifidobacterium lactus and streptococcus thermophilus, and the protease enzymes are comprised of protease serrapeptase, papain, bromelain, amaylase, lipase, rutin and amla.

In yet another embodiment of the present invention, the effective amount of the digestive enzyme provided is 2000 mg to 4000 mg daily, the effective amount of soluble and insoluble fiber provided is 2.0 g to 20 g daily, the effective amount of a mild herbal laxative provided is 900 mg to 6000 mg daily, the effective amount of the probiotic provided provides at least 10 billion microorganisms to the human daily, the effective amount of vitamin C and potassium provided is 2000 mg to 6000 mg of vitamin C and 500 mg to 1200 mg of potassium daily, the effective amount of one or more protease enzymes provided is 1000 mg to 2500 mg of protease enzymes daily, the effective amount of lipase, lysine, taurine, proline, choline, inositol, inositol hexaphosphate, and policosanol provided is 300 mg to 1600 mg of lipase, 50 mg to 600 mg of lysine, 50 mg to 600 mg of taurine, 50 mg to 600 mg of proline, 20 mg to 300 mg of choline, 10 mg to 100 mg of inositol, 10 mg to 100 mg of inositol hexaphosphate and 3 mg to 60 mg of policosanol daily, the effective amount of charcoal, bentonite clay and thyme provided is 0.25 g to 4.50 g of charcoal, 0.25 g to 4.50 g bentonite clay and 0.05 g to 0.75 g of thyme daily, the effective amount of magnesium, potassium, ester-c, cayenne pepper, ginger and niacin provided is 800 mg to 2500 mg of magnesium, 10 mg to 200 mg of potassium, 50 mg to 600 mg of ester-c, 10 mg to 200 mg of niacin and 10 mg to 600 mg of cayenne pepper and 10 mg to 600 mg of ginger daily, the effective amount of lysine is provided by 2 mg to 500 mg of monohydrochloride daily, the effective amount of olive leaf and yarrow flower provided is 1.0 g to 10 g of olive leaf and 1.0 g to 10 g of yarrow flower daily and the effective amount of slippery elm bark provided is 50 mg to 2500 mg daily.

In yet another embodiment, the methods, systems and formulations of the present invention also include an effective amount of sea salt, sodium bicarbonate, bentonite clay and ginger combined together to be applied to the skin of the human using the system at least twice a week.

In yet another embodiment, the methods, systems and formulations of the present invention also include an effective amount of a nutritional meal replacement and flax seed oil, which are to be consumed by a human.

In a detailed aspect of at least one embodiment of the present invention, the nutritional meal replacement is comprised of whey protein isolates, stabilized rice polishings, mineral whey, lecithin, barley grass, wheat grass juice and stevia powder.

In another detailed aspect of at least one embodiment of the present invention, the effective amount of the nutritional meal replacement provided is 7 g to 15 g per meal and the effective amount of flax seed oil provided is 5 ml to 120 ml daily.

In yet another embodiment, the methods, systems and formulations of the present invention also include an effective amount of any combination of at least ten of the following components: para-amino benzoic acid, choline, inositol, vitamin A, vitamin D, vitamin K, vitamin B1, vitamin B3, vitamin B6, vitamin B12, folic acid, biotin, vitamin B5, citrus bioflavonoids, hesperidin, rutin, alfalfa, beet juice, grape seed extract and pine bark, which are to be consumed by a human.

In yet another detailed aspect of at least one embodiment of the present invention, the effective amount of the folic acid provided is 200 mcg to 1500 mcg daily, the effective amount of the biotin provided is 50 mcg to 600 mcg daily, the effective amount of para-amino benzoic acid provided is 5 mg to 40 mg daily, the effective amount of choline provided is to 10 mg to 300 mg daily, the effective amount of inositol provided is 5 mg to 150 mg daily, the effective amount of vitamin A provided is 2000 IU to 20000 IU daily, the effective amount of the vitamin D provided is 50 IU to 800 IU daily, the effective amount of the vitamin K provided is 10 mcg to 200 mcg daily, the effective amount of the citrus bioflavanoids provided is 50 mg to 800 mg daily, the effective amount of the hesperidin provided is 20 mg to 400 mg daily, the effective amount of rutin provided is 5 mg to 60 mg daily, the effective amount of the alfalfa provided is 100 mg to 1000 mg daily, the effective amount of the beet juice provided is 200 mg to 2000 mg daily, the effective amount of the grape seed extract provided is 5 mg to 100 mg daily, the effective amount of vitamin B1 provided is 20 mg to 400 mg daily, the effective amount of the vitamin B2 provided is 20 mg to 400 mg daily, the effective amount of the vitamin B3 is 20 mg to 400 mg daily, the effective amount of the vitamin B6 provided is 10 mg to 400 mg daily, the effective amount of the vitamin B12 provided is 10 mcg to 400 mcg daily, the effective amount of the pine bark provided is 5 mg to 100 mg daily and the effective amount of the vitamin B5 provided is 20 mg to 600 mg daily.

In yet another embodiment, the methods, systems and formulations of the present invention also include an effective amounts of kelp, equisetum arvense, manganese and alfalfa grass, vitamin E, CoQ-10, calcium, magnesium, methylsulfonylmethane, glucosamine HCL, equisetum arvense, harpagophytum procumbens, avena sativa and chromium, which is to be consumed by a human.

In yet another detailed aspect of at least one embodiment of the present invention, the effective amount of the vitamin E provided is 400 IU to 1200 IU of vitamin E daily; the effective amount of CoQ-10 provided is 10 mg to 100 mg daily, the effective amount of manganese provided is 2 mg to 70 mg of manganese daily, the effective amount of calcium provided is 100 to 1000 mg of calcium daily, the effective amount of magnesium provided is 3 mg to 50 mg of magnesium daily, the effective amount of methylsulfonylmethane provided is 30 mg to 500 mg of methylsulfonylmethane daily and the effective amount of glucosamine HCL provided is 30 mg to 500 mg of glucosamine HCL.

In yet another embodiment of the present invention a method of normalizing and improving human body chemistry and the body's natural ability to heal itself is provided. The method comprises providing effective amounts of a digestive enzyme, soluble and insoluble fiber, a laxative, one or more probiotics, vitamin C, potassium, protease enzymes, lipase, lysine, taurine, proline, choline, inositol, inositol hexaphosphate, policosanol, charcoal, bentonite clay, thyme, ascorbic acid, magnesium citrate, methylsulfonyl methane, potassium, ester-c, ginger, niacin, lysine, calcium, stevia leaf, citric acid, a tincture of bayberry bark, juniper berries, yam root, cramp bark, golden seal root, fennel seed, uva ursi leaves, ginger root, lobelia herb, catnip herb, and peppermint leaf, golden seal root, Echinacea angustifolia root, licorice root, a tincture of black walnut hulls, venus fly trap, chaparral, wormwood, licorice root, slippery elm, cloves and comfrey root, and one or more of the following: burdock root, sheep sorrel, rhubarb root, and slippery elm, olive leaf and yarrow flower.

### DETAILED DESCRIPTION

The present invention provides, among other things, a system of supporting, improving and normalizing the body's chemistry so that the body can self-heal and self-regulate.

It is generally accepted that there are five major systems or pathways throughout the human body that are critical to proper body chemistry function and assist with the body's natural ability to heal itself. These pathways are: the intestinal tract, the blood stream, the lymphatic system, the nervous system, and the meridians. Any blockage or reduced flow within any one of these pathways establishes the starting point for disease. The methods, systems and formulations of the present invention allow, among other things, the innate wisdom and natural capabilities of the body to unblock and increase the flow within these pathways, which allows the body to then normalize its natural functions and heal itself.

It is believed that the method, systems of formulations of the present invention create or provide the most ideal internal environment for the human body and the ideal conditions that will allow any form of treatment of disease or illness to work much more effectively.

Because of the universal focus on the treatment of symptoms and disease, the industry standard for both standard western medical practice as well as for what is now commonly termed as alternative medicine, are quite similar in their approach. Both directly address the issue of the patient or client's complaint. With possibly a few exceptions, the cause behind the complaint isn't considered. As a result, only the treatment that has proven the most effective at addressing the complaint, and not its cause, is used to provide relief for the individual. Because only the symptoms and not the cause are addressed, the individual seeking help is often required to follow a treatment plan for an extended if not indefinite period of time. Often times the treatment is not very effective at treating all the patients' complaints effectively and often becomes less effective at treating the patients' complaints over time. As a result, the patients are often forced to seek alternative therapies and assistance for their complaint and/or its underlying condition.

The embodiments of the present invention provide the most ideal internal environment and conditions, both in terms of body chemistry and otherwise, to assist with and improve the body's natural ability to heal itself. Accordingly, the embodiments of the present invention act as powerful methods, systems and formulations for improving and/or normalizing body chemistry functions and the body's natural ability to heal.

In brief and general terms, the embodiments of the present invention include, but are not limited to methods, systems and formulations which act to normalize and/or improve various human body chemistry functions thereby supporting and enhancing the body's innate ability to self-heal and self-regulate to resolve or improve the condition of unwanted disease and illnesses.

More specifically, in one embodiment of the present invention, a system for normalizing and improving human body chemistry and the body's natural ability to heal itself is provided. The system is comprised of effective amounts of a digestive enzyme, soluble and insoluble fiber, a laxative, one or more probiotics, vitamin C, potassium, one or more protease enzymes, lipase, lysine, taurine, proline, choline, inositol, inositol hexaphosphate, policosanol, charcoal, bentonite clay, thyme, ascorbic acid, magnesium citrate, calcium citrate, methylsulfonyl methane, magnesium, potassium, ester-c, ginger and niacin, lysine calcium, stevia leaf, citric acid, a tincture of bayberry bark, juniper berries, yam root, cramp bark, golden seal root, fennel seed, uva ursi leaves, ginger root, lobelia herb, catnip herb, and peppermint leaf, golden seal root, Echinacea angustifolia root, ginger root, and licorice root, a tincture of black walnut hulls, venus fly trap, chaparral, wormwood, licorice root, slippery elm, cloves and comfrey root, burdock root, sheep sorrel, rhubarb root, slippery elm, olive leaf and yarrow flower, which are to be consumed by a human to normalize and improve human's body chemistry and natural ability to heal itself.

In another embodiment of the present invention, the digestive enzyme is obtained from a plant and comprised of any combination of one or more of the following: amylase, protease, lipase, cellulase, maltase, lactase and sucrase enzymes, the soluble and insoluble fiber is comprised of organic flax seed, psyillium seed and husks and oat fibers, the mild herbal laxative is any combination of two or more of the following: senna leaf, cascara sagrada, aloe vera, buckhorn bark, rhubarb root, hyssop leaf and fenugreek seed, the probiotic consists of any combination of five of more of following bacteria or yeast: lactobacillus acidophilus, bifidobacteria bifidum, lactobacillus sporogenes, lactococcus lactus, lactococcus cremoris, lactobacillus kefir, leuconostoc cremoris, lactobacillus caucasicus, lactobacillus plantarum, lactobacillus salivarius, lactobacillus bulgaricus, lactobacillus helveticus, bifidobacterium infantis, bifidobacterium lactus and streptococcus thermophilus, and the protease enzymes are comprised of protease serrapeptase, papain, bromelain, amaylase, lipase, rutin and amla.

In yet another embodiment of the present invention, the effective amount of the digestive enzyme provided is 2000 mg to 4000 mg daily, the effective amount of soluble and insoluble fiber provided is 2.0 g to 20 g daily, the effective amount of a mild herbal laxative provided is 900 mg to 6000 mg daily, the effective amount of the probiotic provided provides at least 10 billion microorganisms to the human daily, the effective amount of vitamin C and potassium provided is 2000 mg to 6000 mg of vitamin C and 500 mg to 1200 mg of potassium daily, the effective amount of one or more protease enzymes provided is 1000 mg to 2500 mg of protease enzymes daily, the effective amount of lipase, lysine, taurine, proline, choline, inositol, inositol hexaphosphate, and policosanol provided is 300 mg to 1600 mg of lipase, 50 mg to 600 mg of lysine, 50 mg to 600 mg of taurine, 50 mg to 600 mg of proline, 20 mg to 300 mg of choline, 10 mg to 100 mg of inositol, 10 mg to 100 mg of inositol hexaphosphate and 3 mg to 60 mg of policosanol daily, the effective amount of charcoal, bentonite clay and thyme provided is 0.25 g to 4.50 g of charcoal, 0.25 g to 4.50 g bentonite clay and 0.05 g to 0.75 g of thyme daily, the effective amount of magnesium, potassium, ester-c, cayenne pepper, ginger and niacin provided is 800 mg to 2500 mg of magnesium, 10 mg to 200 mg of potassium, 50 mg to 600 mg of ester-c, 10 mg to 200 mg of niacin and 10 mg to 600 mg of cayenne pepper and 10 mg to 600 mg of ginger daily, the effective amount of lysine is provided by 2 mg to 500 mg of monohydrochloride daily, the effective amount of olive leaf and yarrow flower provided is 1.0 g to 10 g of olive leaf and 1.0 g to 10 g of yarrow flower daily and the effective amount of slippery elm bark provided is 50 mg to 2500 mg daily.

In yet another embodiment, the methods, systems and formulations of the present invention also include an effective amount of sea salt, sodium bicarbonate, bentonite clay and ginger combined together to be applied to the skin of the human using the system at least twice a week.

In yet another embodiment, the methods, systems and formulations of the present invention also include an effective amount of a nutritional meal replacement and flax seed oil, which are to be consumed by a human.

In a detailed aspect of at least one embodiment of the present invention, the nutritional meal replacement is comprised of whey protein isolates, stabilized rice polishings, mineral whey, lecithin, barley grass, wheat grass juice and stevia powder.

In another detailed aspect of at least one embodiment of the present invention, the effective amount of the nutritional meal replacement provided is 7 g to 15 g per meal and the effective amount of flaxseed oil provided is 5 ml to 120 ml daily.

In yet another embodiment, the methods, systems and formulations of the present invention also include an effective amount of any combination of at least ten of the following components: para-amino benzoic acid, choline, inositol, vitamin A, vitamin D, vitamin K, vitamin B1, vitamin B3, vitamin B6, vitamin B12, folic acid, biotin, vitamin B5, citrus bioflavonoids, hesperidin, rutin, alfalfa, beet juice, grape seed extract and pine bark, which are to be consumed by a human.

In yet another detailed aspect of at least one embodiment of the present invention, the effective amount of the folic acid provided is 200 mcg to 1500 mcg daily, the effective amount of the biotin provided is 50 mcg to 600 mcg daily, the effective amount of para-amino benzoic acid provided is 5 mg to 40 mg daily, the effective amount of choline provided is to 10 mg to 300 mg daily, the effective amount of inositol provided is 5 mg to 150 mg daily, the effective amount of vitamin A provided is 2000 IU to 20000 IU daily, the effective amount of the vitamin D provided is 50 IU to 800 IU daily, the effective amount of the vitamin K provided is 10 mcg to 200 mcg daily, the effective amount of the citrus bioflavanoids provided is 50 mg to 800 mg daily, the effective amount of the hesperidin provided is 20 mg to 400 mg daily, the effective amount of rutin provided is 5 mg to 60 mg daily, the effective amount of the alfalfa provided is 100 mg to 1000 mg daily, the effective amount of the beet juice provided is 200 mg to 2000 mg daily, the effective amount of the grape seed extract provided is 5 mg to 100 mg daily, the effective amount of vitamin B1 provided is 20 mg to 400 mg daily, the effective amount of the vitamin B2 provided is 20 mg to 400 mg daily, the effective amount of the vitamin B3 is 20 mg to 400 mg daily, the effective amount of the vitamin B6 provided is 10 mg to 400 mg daily, the effective amount of the vitamin B12 provided is 10 mcg to 400 mcg daily, the effective amount of the pine bark provided is 5 mg to 100 mg daily and the effective amount of the vitamin B5 provided is 20 mg to 600 mg daily.

In yet another embodiment, the methods, systems and formulations of the present invention also include an effective amounts of kelp, equisetum arvense, manganese and alfalfa grass, vitamin E, CoQ-10, calcium, magnesium, methylsulfonylmethane, glucosamine HCL, equisetum arvense, harpagophytum procumbens, avena sativa and chromium, which is to be consumed by a human.

In yet another detailed aspect of at least one embodiment of the present invention, the effective amount of the vitamin E provided is 400 IU to 1200 IU of vitamin E daily; the effective amount of CoQ-10 provided is 10 mg to 100 mg daily, the effective amount of manganese provided is 2 mg to 70 mg of manganese daily, the effective amount of calcium provided is 100 to 1000 mg of calcium daily, the effective amount of magnesium provided is 3 mg to 50 mg of magnesium daily, the effective amount of methylsulfonylmethane provided is 30 mg to 500 mg of methylsulfonylmethane daily and the effective amount of glucosamine HCL provided is 30 mg to 500 mg of glucosamine HCL.

In yet another embodiment of the present invention a method of normalizing and improving human body chemistry and the body's natural ability to heal itself is provided. The method comprises providing effective amounts of a digestive enzyme, soluble and insoluble fiber, a laxative, one or more probiotics, vitamin C, potassium, protease enzymes, lipase, lysine, taurine, proline, choline, inositol, inositol hexaphosphate, policosanol, charcoal, bentonite clay, thyme, ascorbic acid, magnesium citrate, calcium citrate, methylsulfonyl methane, potassium, ester-c, ginger, niacin, lysine, calcium, stevia leaf, citric acid, a tincture of bayberry bark, juniper berries, yam root, cramp bark, golden seal root, fennel seed, uva ursi leaves, ginger root, lobelia herb, catnip herb, and peppermint leaf, golden seal root, Echinacea angustifolia root, licorice root, a tincture of black walnut hulls, venus fly trap, chaparral, wormwood, licorice root, slippery elm, cloves and comfrey root, and one or more of the following: burdock root, sheep sorrel, rhubarb root, and slippery elm, olive leaf and yarrow flower.

In yet another embodiment, the digestive enzyme is a full spectrum, plant-sourced, before meal digestive enzyme. The primary purpose of the digestive enzymes disclosed herein is to normalize the intestinal tract and enhance the body's ability to digest the food that is ingested.

In yet another embodiment of the present invention, the digestive enzymes disclosed herein substituted for or supplemented with papain, bromelain, hydrochloric acid, or any of the many before-meal digestive aids available on the market.

In yet another embodiment of the present invention, the digestive enzyme is a product called Primagest (Inneractive Nutrition, Costa Mesa, Calif., 714-689-3851). In one particular aspect of the invention 2 Primagest capsules are taken before each meal. It is believed that Primagest is a strong balance of the plant extracted form of Amylase, Protease, Lipase, Cellulase, Maltase, Lactase, and Sucrase in a base blend of Papaya powder and Amla (Indian Gooseberry powder), which act to support the body's ability to digest a full variety of foods contained in a meal.

In yet another embodiment of the present invention, the soluble and insoluble nutritional fiber is provided by a product called Baltone (Inneractive Nutrition, Costa Mesa, Calif., 714-689-3851), which acts as a balanced blend of soluble and insoluble nutritional fiber, along with slippery elm. In at least one embodiment of the present invention, two tablespoons of Baltone are used as part of the system daily to assist in the normalization of the intestinal tract.

It should be appreciated that a high fiber diet eliminates intestinal stagnation and in many ways improves the overall health of the body. When the intestinal tract is sluggish, a supplemental fiber blend is generally recommended. In order to assist the body in normalizing the function of the intestinal tract, a formula is needed that is mild enough yet effective enough to be used on a daily basis until the peristaltic function of the intestinal tract can be restored. It should also be appreciated that while many supplemental fiber blends work well to clear the intestinal tract; most are designed for short-term use. Accordingly, at least in one embodiment of the present invention, a soluble and insoluble nutritional fiber (e.g., Baltone) is used for an extended period of time until the normal function of the intestinal tract is restored.

In yet another embodiment of the present invention, the laxative is a mild herbal laxative found in a product called Fiber Regulator (Inneractive Nutrition, Costa Mesa, Calif., 714-689-3851). Each serving of two capsules of Fiber Regulator contains, among other things, 270 mg. of Senna leaf powder. Fiber Regulator also contains whole leaf aloe vera powder, Buckthorn Bark, Rhubarb root, Hyssop Leaf, Fenugreek Seed, and Aloe Vera gel powder.

It is believed that all laxatives work by mildly irritating the intestinal tract to cause it to expel its contents. It should be appreciated that any mild laxative will fulfill the function of Fiber Regulator and normalizing the function of the intestinal tract without causing a dependency or causing harm to the tissues of the intestinal tract.

In yet another embodiment of the present invention, one to three capsules of Fiber Regulators are used twice daily, in conjunction with soluble and insoluble nutritional fiber (e.g., Baltone). By taking Fiber Regulator at the same time as soluble and insoluble nutritional fiber, the laxative effect of Fiber Regulator causes the intestinal tract to contract against the bulk of the soluble and insoluble nutritional fiber, which provides a resistance to the contraction, thereby creating a toning type of exercise for the muscular sleeve of the intestinal tract rather than just rapidly evacuating the content of the intestinal tract. This method turns the laxative formula into a regulator for the movement of the soluble and insoluble nutritional fiber, to be increased or decreased according to the movement of the individual's bowel.

In yet another embodiment, the probiotic is a product called Nutri-Dophilus (Inneractive Nutrition, Costa Mesa, Calif., 714-689-3851). It consists of a blend of 15 strains of friendly microorganisms. It is believed that the widespread use of chlorinated water and anti-biotics causes the natural balance of the intestinal flora of most every adult colon to be seriously imbalanced. Since it is the friendly or good bacteria that neutralizes the toxic waste generated by the natural presence of unfriendly bacteria, when there is not enough of the friendly or good organisms to neutralize toxic substances, they are drawn into the blood stream and cause many problems and symptoms. It is believed that this imbalance is one of the most common causes of fatigue and play a significant role in the breakdown of the immune system and the most common source of toxic waste that overworks an already congested liver. Accordingly, the addition of an effective probiotic (e.g., Nutri-Dophilus) assists with normalizing and improving body chemistry functions and the body's ability to heal itself. It is believed that a serving of two capsules of Nutri-Dophilus provides over 16 billion mostly friendly or good organisms to the human consuming them. By taking Nutri-Dophilus along with soluble and insoluble nutritional fiber (e.g., Baltone), soluble and insoluble nutritional fiber provides a natural environment for the microorganisms to reproduce within, greatly improving their chances of effectively rebalancing the intestinal flora.

In yet another embodiment of the present invention, the combination of sea salt, baking soda, bentonite clay and ginger is provided by a product called Detox Spa (Inneractive Nutrition, Costa Mesa, Calif., 714-689-3851). In a particular aspect of at least one embodiment of the invention a 20 ounce blend of Detox Spa is provided for external use in a bath.

The body uses the process of perspiration as the method of removing toxic chemicals, metals, and acid waste through the skin, which is also the largest organ of elimination of the body. There have been several forms of detoxification formulation for use in baths developed over time. It is believe that Detox Spa works to normalize and improve body chemistry and the body's ability to heal in numerous ways. First, it is believed that the ginger stimulates a greater opening of the pores in the skin to allow larger volumes of waste to be removed. The sea salt in Detox Spa works by osmosis to increase the movement of water from inside the body to the outside, thereby improving the detoxification efforts of perspiration. The bentonite clay works in the same manner as a poultice. Because clay has a strong pulling effect, especially on metals, it removes them from the body when applied to the skin. Baking soda has the effect of neutralizing the acid waste being eliminated, preventing skin irritation. The high mineral content of the entire formula also enhances electron transfer in the water to allow the body to release any excess radiation that might have been stored in the body. In one aspect of the present invention, the Detox Spa product is used two times every week to remove waste products and normalize or improve body chemistry and the body's ability to heal.

In yet another embodiment of the present invention, the nutritional meal replacement is provided by a product called Essential Light (Inneractive Nutrition, Costa Mesa, Calif., 714-689-3851). Essential Light is a whole food nutritional blend, used as a meal replacement. Essential Light provides a concentrated form of nutrients that requires little to no energy to break it down and eliminate its waste. It should be appreciated that other nutritional meal replacements with approximately the same nutritional value will provide the same or similar benefits. In yet another detailed aspect of the invention, Essential Light is taken with an effective amount of flax seed oil to provide a very healthy balance of protein, carbohydrate, and fat, as well as food enzymes, vitamins and minerals. More specifically, two tablespoons of Essential Light is combined with one tablespoon of flax oil in a milk replacement (e.g., almond milk or soymilk) as a nutritional meal replacement. In yet another aspect of the present invention, this nutritional meal replacement may be taken one or two times a day.

In another embodiment of the present invention, one or two tablespoons of flax seed oil is taken per day. It is believed that flax seed oil has a greater amount of the omega 3 than the omega 6 fatty acids. The flax seed oil can be used in combination with other aspects of the systems and formulations disclosed herein to improve and normalize body chemistry and the body's ability to heal itself.

In yet another embodiment of the present invention, the para-amino benzoic acid, choline, inositol, vitamin A, vitamin D, vitamin K, vitamin B1, vitamin B3, vitamin B6, vitamin B12, folic acid, biotin, vitamin B5, citrus bioflavonoids, hesperidin, rutin, alfalfa, beet juice, grape seed extract and pine bark is provided by a product called Metabal (Inneractive Nutrition, Costa Mesa, Calif., 714-689-3851). Metabal is an intentionally incomplete multi-vitamin. Metabal contains, among other things, a good balance of B vitamins, folic acid, biotin, paba; along with choline, inositol, vitamins A, D, and K. Metabal also contains citrus bioflavonoids, hesperidin and rutin, to further enhance the food sourced vitamin C. In one detailed aspect of the invention, four capsules of Metabal are provided or taken by the human each day.

In yet another embodiment of the present invention, the kelp, equisetum arvense (a.k.a. Horsetail herb), manganese and alfalfa grass is provided by a product called Complemin (Inneractive Nutrition, Costa Mesa, Calif., 714-689-3851). Complemin is a combination of Kelp, Horsetail herb and alfalfa grass juice powder, with an additional 7.9 mg of Manganese per serving. Complemin provides a comfortable amount of organically derived major minerals such as calcium, magnesium, sodium and potassium, as well as the many trace minerals such as selenium, copper, zinc, and manganese. In yet another embodiment of the present invention, four capsules of Complemin are provided or consumed by the human daily.

In yet another embodiment of the present invention, acerola cherry powder, ester-c, magnesium and potassium is provided by a produced called Cyntegrity (Inneractive Nutrition, Costa Mesa, Calif., 714-689-3851). In another detailed aspect of at least one embodiment of the present invention, four capsules of Cyntegrity are provided to or taken by the human each day.

For decades ascorbic acid has proven itself as a great boost to the immune system. Unfortunately, ascorbic acid is only one of the many important components of a complete form of vitamin C. When we consume a complete food-source of vitamin C we receive the bioflavonoids, hesperidin, rutin, and many other components along with a natural form of ascorbic acid. These other components are essential to the complete health of the human body. These other essential components, many of which are found in Cyntegrity, are needed to maintain the integrity of the glands, arteries, and skin, as well as the overall integrity of the entire body. When a person consumes only ascorbic acid as their source of vitamin C, they suffer from a deficiency of these other nutrients and subtle symptoms of this deficiency continue to affect the overall health of the entire body.

As disclosed, various embodiments of the present invention include vitamin E, which has been successfully used for decades to neutralize the destructive effects of free radicals, thereby greatly reducing tissue destruction. Vitamin E has also been used to improve the efficiency of oxygen in the cells of the body. In another detailed aspect of at least one embodiment of the present invention, a total of 800 IUs of vitamin E are provided or taken by a human each day.

In yet another embodiment of the present invention, the calcium, methylsulfonyl methane, glucosamine HCL, equisetum arvense (horsetail), harpagophytum procumbens (devil's claw), and avena sativa (oat straw) are provided by a product called Coral Calcium (Inneractive Nutrition, Costa Mesa, Calif., 714-689-3851). The formula is designed to provide an easy to assimilate form of calcium along with the other herbs and nutrients that have been found to be supportive of joint and cartilage health. In another detailed aspect of at least one embodiment of the present invention, two capsules of Coral Calcium are taken each day for various health reasons, including general bone, joint and cartilage support.

In yet another embodiment of the present invention, the CoQ-10: is provided by a product called CoQ-10 (Inneractive Nutrition, Costa Mesa, Calif., 714-689-3851), which is a combination of coenzyme Q-10, vitamin A, and vitamin E in a base of rice bran oil. Unfortunately, most adult bodies are proving themselves to be incapable of producing this needed coenzyme. Therefore it is being found to be a great enhancement to the function of the body to supplement this vital co-enzyme. CoQ-10 is critical in the production of energy within each cell of the body. It is also essential for the overall health of every cell within the human body. In yet another detailed aspect of at least one embodiment of the present invention, one to two capsules of CoQ-10 are provided to or taken by a human daily.

In yet another embodiment of the present invention, the chromium is provided by a product called Chromium (Inneractive Nutrition, Costa Mesa, Calif., 714-689-3851). Chromium provides 200 mcg of chromium from vegetable culture per serving. Most adults today are at least fourth generation sugar abusers. Since chromium is an essential component of the glucose tolerance factor, and since the abuse of white sugar is the cause for chromium depletion within the body, most adults are found to be extremely deficient of the trace mineral chromium. Chromium is an essential trace element for animals and man. Chromium, being a key ingredient in the body's production of the Glucose Tolerance Factor (GTF) makes it absolutely essential for several reasons to replace this deficient element in the body. GTF stimulates insulin activity, thereby assisting the body in metabolizing sugar, or glucose. Without it, insulin cannot adequately control blood sugar levels. It also helps to control fat levels and cholesterol levels in the blood. In another detailed aspect of at least one embodiment of the present invention, two capsules of 200 mcg of chromium are provided to or taken by a human daily.

In yet another embodiment of the present invention, lipase, lysine, taurine, proline, choline, inositol, inositol hexaphosphate, and policosanol are provided by a product called Cholesterall Clear (Inneractive Nutrition, Costa Mesa, Calif., 714-689-3851), which provides a combination of lipase, lysine, taurine, proline, choline, inositol, inositol hexaphosphate (a.k.a. IP6), and policosanol. Individually each of these ingredients has been found to have a dissolving effect on the plaque type materials that accumulate on the walls of the arteries throughout the human body. Collectively this formula has been designed to assist the body's natural ability to break down this substance and release it from the body. It is believed that this formula effectively enhances the body's efforts to dissolve plaque type materials, not only from the arteries, but from the gallbladder and the liver as well. In another preferred embodiment of the present invention, two capsules of Cholesterol Clear are provided to or taken by a human daily.

In yet another embodiment of the present invention, the protease enzymes are provided by a product called Vitalzime (Inneractive Nutrition, Costa Mesa, Calif., 714-689-3851). Vitalzyme is a protease enzyme combination. Because of the intensified detoxification taking place as a result of the other aspects of the methods, systems and formulations disclosed herein, it is important that as much energy as the body can make available be directed toward the efficient and complete removal of all toxic debris that has accumulated within the body. Since various types of inflammation are quite common within the average adult body, it is a significant enhancement to assist the body in eliminating as much inflammation as possible. This will allow the energy that would normally be directed towards a continued effort to reduce the inflammation, to be redirected into the detoxification process. Because an excess of unusable protein residue is generally found associating with various forms of inflammation, the use of a strong proteolytic enzyme has been shown to greatly reduce many conditions of inflammation. Therefore, if a supplemental enzyme can perform the work that would require a considerable amount of energy for the body to accomplish, to use an enzyme to accomplish this work would free up the much needed energy for other accomplishments. In another detailed aspect of at least one embodiment of the present invention, at least four capsules of Vitalzyme are provided to or consumed by a human daily.

In yet another embodiment of the present invention, the charcoal, bentonite clay and thyme are provided by a product called Detox Relief (Inneractive Nutrition, Costa Mesa, Calif., 714-689-3851). Detox Relief assists with absorption of toxic chemicals and gas from the intestinal tract so the body can safely eliminate these substances out through the bowel. Both clay and charcoal have been used traditionally for decades to absorb gas and toxic chemicals from the intestinal track. In another detailed aspect of at least one embodiment of the present invention, six capsules of Detox Relief are provided to or taken by a human.

In yet another embodiment of the present invention, the ascorbic acid, magnesium citrate, calcium citrate, methylsulfonyl methane is provided by a product called C-Tox (Inneractive Nutrition, Costa Mesa, Calif., 714-689-3851). Ascorbic acid has been found to possess a chelating effect on toxic metals. Ascorbic Acid will bind to metals, allowing the body a better opportunity of eliminating them. This formula is believed to bind the toxic metals being released from the gallbladder into the intestinal tract, to enhance the cleansing of the intestinal tract, and provide a healing benefit for the tissues of the intestinal tract. In another detailed aspect of at least one embodiment of the present invention, at least four capsules of C-Tox are provided to or taken by a human daily.

In yet another embodiment of the present invention, the magnesium, ascorbic acid, ester-c, cayenne pepper, ginger and niacin is provided by a product called Second Breath (Inneractive Nutrition, Costa Mesa, Calif., 714-689-3851). Second Breath enhances the cleansing of the intestinal tract. In another detailed aspect of at least one embodiment of the present invention, four capsules of Second Breath are provided to or taken by a human daily.

In yet another embodiment of the present invention, the calcium, stevia leaf extract, and citric acid is provided by a product called Re-Hydrating Elements of Life (Inneractive Nutrition, Costa Mesa, Calif., 714-689-3851). Re-Hydrating Elements of Life is a combination of organic and ionized minerals, calcium, stevia leaf extract, and citric acid in a base of water. In another detailed aspect of at least one embodiment of the present invention, three tablespoons of Re-Hydrating Elements of Life is provided to or taken by the human daily.

In yet another embodiment of the present invention, the tincture of bayberry bark, juniper berries, wild yam root, cramp bark, golden seal root, fennel seed, uva ursi leaves, ginger root, lobelia herb, catnip herb, and peppermint leaf is provided by a product called Liver and Kidney Support (Inneractive Nutrition, Costa Mesa, Calif., 714-689-3851). The tincture is drawn with pure vodka. It is believed that each of the ingredients has been used either by itself or in combination with at least one other of the other herbs in the formula to assist the function of the liver and/or kidneys and together they greatly enhance the detoxification of body the liver and the kidneys, as well as to strengthen both organ systems. In another detailed aspect of at least one embodiment of the present invention, three servings of three droppers of Liver and Kidney Support is provided to or taken by a human daily.

In yet another embodiment of the present invention, the golden seal root, Echinacea angustifolia root, ginger root, and licorice root is provided by a product called Immune Enhancer (Inneractive Nutrition, Costa Mesa, Calif., 714-689-3851). Immune Enhancer. It is believed that Immune Enhancer greatly improves the body's ability to rid itself of viruses, bacteria, fungi, parasites and weak or mutated cells. In another detailed aspect of at least one embodiment of the present invention, three servings of four droppers of Immune Enhancer are provided to or taken by a human daily.

In yet another embodiment of the present invention, the a tincture drawn from black walnut hulls, venus fly trap, chaparral, wormwood, licorice root, slippery elm, cloves and comfrey root is provided by a product called Especially For Parasites (Inneractive Nutrition, Costa Mesa, Calif., 714-689-3851). Many combinations of the herbs that make up the formula of Especially For Parasites have been used for decades to eliminate parasites from the human body. In one detailed aspect of at least one embodiment of the present invention three servings of three droppers are provided to or taken by a human daily.

In yet another embodiment of the present invention, the burdock root, sheep sorrel, rhubarb root, and slippery elm is provided in a product called Angel Tea (Inneractive Nutrition, Costa Mesa, Calif., 714-689-3851). In anther detailed aspect of at least one embodiment of the present invention, six ounces of the tea made Angel Tea is provided to or taken by a human once or twice daily.

In yet another embodiment of the present invention, the olive leaf and yarrow flower is provided by a product called Olive Leaf (Inneractive Nutrition, Costa Mesa, Calif., 714-689-3851). Olive Leaf contains both olive leaf and yarrow flower. In another detailed aspect of at least one embodiment of the present invention, nine capsules of Olive Leaf are provided to or taken by a human daily.

Numerous individuals who followed the methods, systems and formulations disclosed herein experienced improved general health and energy, a normalization or improvement in their body's chemistry and ability to heal itself. Some experienced a complete disappearance of their serious health conditions.

It should be appreciated that the embodiments of the present disclosure can be provided in any form or method known in the art. It should also be appreciated that an effective dose of the items disclosed will depend on the individual taking the doses and their particular condition. Unless otherwise stated, effective doses should be considered typical doses, which are well known in the art, modified accordingly to take into consideration relevant factors (e.g., age, weight, sex and condition) of the human taking it.

### EQUIVALENTS OF THE INVENTION

While particular embodiments of the invention and variations thereof have been described in detail, other modifications and methods of using the disclosed methods, systems and therapeutic formulations will be apparent to those of skill in the art. Accordingly, it should be understood that various applications, modifications, and substitutions may be made of equivalents without departing from the spirit of the invention or the scope of the claims. Various terms have been used in the description to convey an understanding of the invention; it will be understood that the meaning of these various terms extends to common linguistic or grammatical variations or forms thereof. It will also be understood that when systems, formulations, or compositions have been identified by trade names, product names, or common names, that these names are provided as contemporary examples, and the invention is not limited by such literal scope, particularly when the systems, formulations or compositions have been further described in terms of class or type and/or by mechanism of action. Although the description offers biochemical theory and interpretation, it should be understood that such theory and interpretation do not bind or limit the claims. Further, it should be understood that the invention is not limited to the embodiments that have been set forth for purposes of exemplification, but is to be defined only by a fair reading of the appended claims, including the full range of equivalency to which each element thereof is entitles.

1. A system for normalizing and improving human body chemistry and the body's natural ability to heal itself comprising the following components of the system:
an effective amount of a digestive enzyme to be consumed by a human using the system;
an effective amount of soluble and insoluble fiber to be consumed by a human using the system;
an effective amount of a laxative to be consumed by a human using the system;
an effective amount of one or more probiotics to be consumed by a human using the system;
an effective amount of vitamin C and potassium to be consumed by a human using the system;
an effective amount of one or more protease enzymes to be consumed by a human using the system;
an effective amount of lipase, lysine, taurine, proline, choline, inositol, inositol hexaphosphate, and policosanol to be consumed by a human using the system;
an effective amount of charcoal, bentonite clay and thyme to be consumed by a human using the system;
an effective amount of ascorbic acid, magnesium citrate, calcium citrate and methylsulfonyl methane to be consumed by a human using the system;
an effective amount of magnesium, potassium, ester-c, cayenne pepper, ginger and niacin to be consumed by a human using the system;
an effective amount of lysine to be consumed by a human using the system;
an effective amount of calcium, stevia leaf, and citric acid to be consumed by a human using the system;
an effective amount of a tincture of bayberry bark, juniper berries, yam root, cramp bark, golden seal root, fennel seed, uva ursi leaves, ginger root, lobelia herb, catnip herb, and peppermint leaf to be consumed by a human using the system;
an effective amount of golden seal root, Echinacea angustifolia root, ginger root, and licorice root to be consumed by a human using the system;
an effective amount of a tincture drawn from black walnut hulls, venus fly trap, chaparral, wormwood, licorice root, slippery elm, cloves and comfrey root to be consumed by a human using the system;
an effective amount of any combination of one or more of the following: burdock root, sheep sorrel, rhubarb root, and slippery elm to be consumed by a human using the system; and
an effective amount of olive leaf and yarrow flower to be consumed by a human using the system.

2. The system of claim 1, wherein the digestive enzyme is obtained from a plant and comprised of any combination of one or more of the following: amylase, protease, lipase, cellulase, maltase, lactase and sucrase enzymes; wherein the soluble and insoluble fiber is comprised of organic flax seed, psyillium seed and husks and oat fibers; wherein the mild herbal laxative is comprised any combination of two or more of the following: senna leaf, cascara sagrada, aloe vera, buckhorn bark, rhubarb root, hyssop leaf and fenugreek seed; wherein the probiotic consists of any combination of five of more of following bacteria or yeast: Lactobacillus Acidophilus, Bifidobacteria Bifidum, Lactobacillus Sporogenes, Lactococcus Lactus, Lactococcus Cremoris, Lactobacillus Kefir, Leuconostoc Cremoris, Lactobacillus Caucasicus, Lactobacillus Plantarum, Lactobacillus Salivarius, Lactobacillus Bulgaricus, Lactobacillus Helveticus, Bifidobacterium Infantis, Bifidobacterium Lactus and Streptococcus Thermophilus; and wherein the protease enzymes are comprised of protease serrapeptase, papain, bromelain, amaylase, lipase, rutin and amla.

3. The system of claim 2, wherein the effective amount of the digestive enzyme is 2000 mg to 4000 mg daily; wherein the effective amount of soluble and insoluble fiber is 2.0 g to 20 g daily; wherein the effective amount of a mild herbal laxative is 900 mg to 6000 mg daily; wherein the effective amount of the probiotic provides at least 10 billion microorganisms to the human daily; wherein the effective amount of vitamin C and potassium provides 2000 mg to 6000 mg of vitamin C and 500 mg to 1200 mg of potassium daily; wherein effective amount of one or more protease enzymes provides 1000 mg to 2500 mg of protease enzymes daily; wherein the effective amount of lipase, lysine, taurine, proline, choline, inositol, inositol hexaphosphate, and policosanol provides 300 mg to 1600 mg of lipase, 50 mg to 600 mg of lysine, 50 mg to 600 mg of taurine, 50 mg to 600 mg of proline, 20 mg to 300 mg of choline, 10 mg to 100 mg of inositol, 10 mg to 100 mg of inositol hexaphosphate and 3 mg to 60 mg of policosanol daily; wherein the effective amount of charcoal, bentonite clay and thyme provides 0.25 g to 4.50 g of charcoal, 0.25 g to 4.50 g bentonite clay and 0.05 g to 0.75 g of thyme daily; wherein the effective amount of magnesium, potassium, ester-c, cayenne pepper, ginger and niacin provides 800 mg to 2500 mg of magnesium, 10 mg to 200 mg of potassium, 50 mg to 600 mg of ester-c, 10 mg to 200 mg of niacin and 10 mg to 600 mg of cayenne pepper and ginger daily; wherein the effective amount of lysine is provided by 2 mg to 500 mg of monohydrochloride daily; wherein the effective amount of olive leaf and yarrow flower provides 1.0 to 10 g of olive leaf and 1.0 to 10 g of yarrow flower; wherein the effective amount of slippery elm bark is 50 mg to 2500 mg daily.

4. The system of claim 1 further comprising:
an effective amount of sea salt, sodium bicarbonate, bentonite clay and ginger combined together to be applied to the skin of the human using the system at least twice a week.

5. The system of claim 4 further comprising:
an effective amount of a nutritional meal replacement and flax seed oil to be consumed by a human using the system.

6. The system of claim 5, wherein the nutritional meal replacement is comprised of whey protein isolates, stabilized rice polishings, mineral whey, lecithin, barley grass, wheat grass juice and stevia powder.

7. The system of claim 5, wherein the effective amount of the nutritional meal replacement is 7 g to 15 g per meal; and wherein the effective amount of flaxseed oil is 5 ml to 120 ml daily.

8. The system of claim 5, further comprising:
an effective amount of any combination of ten of the following components: para-amino benzoic acid, choline, inositol, vitamin A, vitamin D, vitamin K, vitamin B1, vitamin B3, vitamin B6, vitamin B12, folic acid, biotin, vitamin B5, citrus bioflavonoids, hesperidin, rutin, alfalfa, beet juice, grape seed extract and pine bark to be consumed by a human using the system.

9. The system of claim 8, wherein the effective amount of the folic acid is 200 mcg to 1500 mcg daily; wherein the effective amount of the biotin is 50 mcg to 600 mcg daily; wherein the effective amount of para-amino benzoic acid is 5 mg to 40 mg daily; wherein the effective amount of choline is 10 mg to 300 mg daily; wherein the effective amount of inositol is 5 mg to 150 mg daily; wherein the effective amount of vitamin A is 2000 IU to 20000 IU daily; wherein the effective amount of the vitamin D is 50 IU to 800 IU daily; wherein the effective amount of the vitamin K is 10 mcg to 200 mcg daily; wherein the effective amount of the citrus bioflavanoids is 50 mg to 800 mg daily; wherein the effective amount of the hesperidin is 20 mg to 400 mg daily; wherein the effective amount of rutin is 5 mg to 60 mg daily; wherein the effective amount of the alfalfa is 100 mg to 1000 mg daily; wherein the effective amount of the beet juice is 200 mg to 2000 mg daily; wherein the effective amount of the grape seed extract is 5 mg to 100 mg daily; wherein the effective amount of the vitamin B1 is 20 mg to 400 mg daily; wherein the effective amount of the vitamin B2 is 20 mg to 400 mg daily; wherein the effective amount of the vitamin B3 is 20 mg to 400 mg daily; wherein the effective amount of the vitamin B6 is 10 mg to 400 mg daily; wherein the effective amount of the vitamin B12 is 10 mg to 400 mg daily; wherein the effective amount of the pine bark is 5 mg to 100 mg daily and wherein the effective amount of the vitamin B5 is 20 mg to 600 mg daily.

10. The system of claim 8, further comprising:
an effective amount of kelp, equisetum arvense, manganese and alfalfa grass to be consumed by a human using the system;
an effective amount of vitamin E to be consumed by a human using the system;
an effective amount of CoQ-10 to be consumed by a human using the system;
an effective amount of calcium, magnesium, methylsulfonylmethane, glucosamine HCL, equisetum arvense, harpagophytum procumbens, and avena sativa to be consumed by a human using the system; and
an effective amount of chromium to be consumed by a human using the system.

11. The system of claim 10, wherein the effective amount of the vitamin E provides 400 IU to 1200 IU of vitamin E daily; wherein the effective amount of CoQ-10 provides 10 mg to 100 mg daily; wherein the effective amount manganese provides 2 mg to 70 mg of manganese daily; wherein the effective amount of calcium provides 100 to 1000 mg of calcium daily, wherein the effective amount of magnesium provides 3 mg to 50 mg of magnesium daily; wherein the effective amount of methylsulfonylmethane provides 30 to 500 mg of methylsulfonylmethane daily; and wherein the effective amount of glucosamine HCL provides 30 to 500 mg of glucosamine HCL.

12. A method of normalizing and improving human body chemistry and the body's natural ability to heal itself comprising:
providing an effective amount of a digestive enzyme to be consumed by a human;
providing an effective amount of soluble and insoluble fiber to be consumed by a human;
providing an effective amount of a laxative to be consumed by a human using the system;
providing an effective amount of one or more probiotics to be consumed by a human using the system;
providing an effective amount of vitamin C and potassium to be consumed by a human;
providing an effective amount of one or more protease enzymes to be consumed by a human;
providing an effective amount of lipase, lysine, taurine, proline, choline, inositol, inositol hexaphosphate, and policosanol to be consumed by a human;
providing an effective amount of charcoal, bentonite clay and thyme to be consumed by a human;
providing an effective amount of ascorbic acid, magnesium citrate, calcium citrate and methylsulfonyl methane to be consumed by a human;
providing an effective amount of magnesium, potassium, ester-c, cayenne pepper, ginger and niacin to be consumed by a human;
providing an effective amount of lysine to be consumed by a human;
providing an effective amount of calcium, stevia leaf, and citric acid to be consumed by a human;
providing an effective amount of a tincture of bayberry bark, juniper berries, yam root, cramp bark, golden seal root, fennel seed, uva ursi leaves, ginger root, lobelia herb, catnip herb, and peppermint leaf to be consumed by a human;
providing an effective amount of golden seal root, Echinacea angustifolia root, ginger root, and licorice root to be consumed by a human;
providing an effective amount of a tincture drawn from black walnut hulls, venus fly trap, chaparral, wormwood, licorice root, slippery elm, cloves and comfrey root to be consumed by a human;
providing an effective amount of any combination of one or more of the following: burdock root, sheep sorrel, rhubarb root, and slippery elm to be consumed by a human; and
providing an effective amount of olive leaf and yarrow flower to be consumed by a human.
